(19) United States
(12) Patent Application Publication
(10) Pub. No.: US 2008/0241944 A1
(43) Pub. Date: Oct. 2, 2008

(54) METHOD OF MAKING GEL DROP PROTEIN BIOCHIPS

(75) Inventor: Daniel S. Schabacker, Naperville, IL (US)

Correspondence Address: BARNES & THORNBURG LLP, P.O. BOX 2786, CHICAGO, IL, 60690-2786, US

(73) Assignee: UCHICAGO ARGONNE, LLC, Chicago, IL (US)

(21) Appl. No.: 11/693,523

(22) Filed: Mar. 29, 2007

(51) Int. Cl. G01N 33/00

(52) U.S. Cl. 436/86

(57) ABSTRACT

A method of making a gel drop protein chip by transferring proteins, which were obtained from a cellular lysate, partitioned using two-dimensional, protein fractionation, and mixed with a polymeric matrix solution containing acrylamide/bis and glycerol, to an array; a method of making a gel drop protein chip by transferring proteins, which were derivatized with N-hydroxysuccinimide ester of N-methacryloyl-6-aminocaproic acid (NHS monomer), and mixed with a polymeric matrix solution containing acrylamide/bis and glycerol, to an array; a gel drop protein chip containing proteins in a polymeric matrix solution containing acrylamide/bis glycerol; a method of using the gel drop protein chip to interrogate a sample; and a protein derivatized with the NHS monomer.

### STATEMENT OF GOVERNMENTAL SUPPORT

This invention was partially conceived under Contract No. W-31-109-ENG-38 between the U.S. Department of Energy and the University of Chicago representing Argonne National Laboratory. This invention was supported, at least in part, with funding from the Department of Energy under Agreement/Award No. 03A18. Therefore, the Government of the United States of America has certain rights in the invention.

### BACKGROUND

Methods and compositions for making a gel drop protein chip are described.

The protein chip is recognized as a desirable tool in drug discovery and proteomics. Advances in protein chip technology, however, have been hindered by the heterogeneous nature of proteins, making multiplexed analysis of proteins substantially more difficult than that of nucleic acids. In addition, sequenced genomes and labor-intensive recombinant expression and purification systems are required to obtain proteins in sufficient amounts for protein array fabrication. Unfortunately, the majority of recombinantly expressed proteins lack post-translational modifications, which, oftentimes, are required for proper function, and expression systems for outer membrane proteins are still in their infancy. Taken together, these issues significantly impact the development of high-density protein arrays for both basic biological and applied diagnostic sciences.

### SUMMARY

A method of making a gel drop protein chip is described. The method includes transferring proteins, which were obtained from a cellular lysate, partitioned using two-dimensional, protein fractionation (PF2D), and mixed with a polymeric matrix solution containing acrylamide/bis and glycerol, to an array. Derivatization is not required.

Another method of making a gel drop protein chip is also described. The method includes transferring proteins, which were derivatized with N-hydroxysuccinimide ester of N-methacryloyl-6-aminocaproic acid (NHS monomer), and mixed with a polymeric matrix solution containing acrylamide/bis and glycerol, to an array.

Also described is a gel drop protein chip. The gel drop protein chip contains proteins in a polymeric matrix solution containing acrylamide/bis and glycerol.

Further described is a method of using the gel drop protein chip. The method includes using the gel drop protein chip to interrogate a sample.

Still further described is a derivatized protein. The protein is derivatized with NHS monomer.

### DETAILED DESCRIPTION

In order to circumvent one of the most significant barriers to protein array fabrication (i.e., limited quantities of functional protein), subcellular isolation is coupled with PF2D to fractionate functional proteins (i.e., post-translational modifications necessary for function are present). The fractionated proteins are then deposited onto a gel element protein array—a biochip platform that addresses probe denaturation and steric hindrance issues typically associated with planar array substrates. “Probe” as defined herein is a molecule on an array that functions as a capture entity. “Target” is a molecule in a sample. Fluid-phase fractionation avoids the need to sequence, clone, and express the entire genome from every organism of interest, circumvents issues of membrane protein precipitation, preserves intact protein structure, provides highly reproducible separations for inter-lysate comparisons among multiple protein sample pools, is amenable to high-throughput analysis, allows significantly higher loading than conventional techniques, while maintaining a high degree of resolution, and, most importantly, providing proteins as expressed by the cells. Use of the gel element protein array increases probe capacity, defines probe volume/area, provides a non-denaturing, liquid-phase environment, and provides even spatial distribution of the probe. Comprehensive, proteome-scale, functional protein arrays (protein biochips, and, more specifically, gel drop protein biochips or GDPBs) result.

In view of the above, provided is a method of making a GDPB. The method includes transferring proteins, which were obtained from a cellular lysate, partitioned using PF2D, and mixed with a polymeric matrix solution containing acrylamide/bis and glycerol, to an array. The proteins optionally can be derivatized with NHS monomer prior to being mixed with the polymeric matrix solution. Alternatively, the method includes transferring proteins, not using PF2D, which were derivatized with NHS monomer, and mixed with a polymeric matrix solution comprising acrylamide/bis and glycerol, to an array.

Any suitable cell sample can be used. The sample can be obtained from a multi-cellular organism, in which case the sample can be obtained from blood, serum, a tissue, or an organ. Alternatively, the sample can be obtained from a unicellular organism, such as a bacterium, or a virus, and the like. Preferably, the cell sample is lysed, and a subcellular fraction (e.g., membrane or cytosol) is obtained. The subcellular fraction can be obtained in accordance with any suitable method as is known in the art. Subcellular fractionation reduces the complexity of the whole cell lysate, such that nearly homogeneous protein fractions can be obtained.

The subcellular fraction then can be subjected to PF2D to fractionate functional proteins. While any suitable method of PF2D can be used, preferably, the PF2D includes partitioning on the basis of isoelectric focusing, and fractionating on the basis of hydrophobicity using reverse phase-separation in liquid phase, such as exemplified herein. Proteomic profiling (analytical) and fraction collecting (preparative) then can be performed.

The GDPBs can be made with derivatized and/or un-derivatized proteins. The proteins themselves, can be well-defined and pure, or they can be undefined fractionates, such as fractionates obtained from PF2D of subcellular fractions of cellular lysates. Derivatization of proteins allows for covalent attachment of the proteins to the matrix of the gel drop and avoids leaching of the proteins during the initial wash of the biochip. Un-derivatized proteins are merely entrapped within the polymeric matrix of the gel drop, and approximately 30-40% of the proteins initially deposited within the polymeric matrix of the gel are lost during the initial wash of the biochip.

The protein can be derivatized with any bi-functional cross-linker that introduces a polymer-reactive species onto the protein and facilitates the attachment of the protein to the polymeric matrix of the gel. When a polymeric matrix solution containing acrylamide/bis and glycerol, such as 5% acrylamide/bis and 65% glycerol (w/v) in 35 mM sodium phosphate, pH 7.25, is used, a preferred cross-linker is the NHS monomer (see U.S. Pat. App. Pub. No. 2005/0042363), which derivatizes proteins via methacrylate. The cross-linker can be prepared and protein can be derivatized in accordance with the methods set forth in Example 1, e.g., with about a 10-fold excess of a 0.5% solution of the NHS monomer in N,N-dimethylformamide (DMF).

The derivatized protein is then placed in the polymeric matrix solution. Likewise, underivatized protein is separately placed in the polymeric matrix solution. Polymeric solution containing either derivatized or underivatized proteins is used for the fabrication of GDPBs. While any suitable methods can be used, examples of methods are set forth in the Examples. When the gel drops are fully hydrated, the gel drops are approximately 150 μm in diameter.

In view of the above, a gel drop protein chip is provided. The gel drop protein chip contains proteins in a polymeric matrix solution containing acrylamide/bis and glycerol.

Also, in view of the above, a method of using the gel drop protein chip is provided. The method includes using the gel drop protein chip to interrogate a sample.

A derivatized protein is also provided. The protein is derivatized with NHS monomer.

### EXAMPLES

The following examples serve to illustrate the present invention. They are not intended to limit the scope of the invention in any way.

### Example 1

This example describes the preparation of a GDPB.

Protein fractionation: A bacterial lysate was fractionated by isoelectric point on a high-performance chromatofocusing (HPCF) column. After isoelectric focusing, the resultant fractions were applied to a nonporous, reverse-phase, high-performance, liquid chromatography column (NPS-RP-HPLC) for fractionation based on hydrophobicity. An automated fractionater was used to deposit the fractions into a 96-deep well plate.

Cross-linker: A 5% solution (50 mg/ml) of NHS monomer (MW approx. 280; Pierce Biotechnology, Inc., Rockford, Ill.) in DMF was prepared. Ten microliters of the solution were added to 90 μl DMF, thereby providing a 0.5% solution of NHS monomer.

Protein derivatization: Antibodies were methacrylated in a 10-fold molar excess of NHS monomer by adding 0.75 μl of 0.5% NHS monomer in DMF to 0.2 mg of antibody. The mixture was vortexed, incubated on ice for 2 hrs while protected from light, and then dialyzed against modified Dulbecco's phosphate-buffered saline (PBS), pH 7.4, overnight while protected from light. The entire volume was removed from dialysis, transferred between two tubes (0.1 mg/tube), and dried in a Speedvac. The methacrylated antibodies were stored desiccated and protected from light at 5° C.

Polymeric matrix solution: A polymeric matrix solution of 5% acrylamide/bis and 65% glycerol (w/v) in 35 mM sodium phosphate, pH 7.25, was prepared.

Derivatized proteins in polymeric matrix solution: The polymeric matrix solution (100 μl) was added to the desiccated, methylacrylated antibodies (0.1 mg) in a microfuge tube, and thoroughly mixed by pipetting up and down. The microfuge tube was centrifuged briefly to collect the solution at the bottom of the tube, allowed to sit for 30 min at room temperature (RT) protected from light, and then transferred to acrylic slides (VACR, Cel Assoc., Inc., Pearland, Tex.) using a Genetix Q-Array pin arrayer (Genetix Ltd., United Kingdom).

Un-derivatized proteins in polymeric matrix solution: Eprogen 96-well plates containing un-derivatized fractionated proteins were dried in a Speedvac. The polymeric matrix solution (50 μl) was added to each well in each plate. The plates were spun for 1 min in two orientations to aid the mixing. The plates were then allowed to incubate at RT for 30 min protected from light. Forty μl from each well were transferred into a separate well of a 384-well Genetix source plate. During the transfer process, the pipette was used to aid in mixing by pipetting the solution repeatedly up and down. The source plates were centrifuged for 1-2 min, and then stored sealed at 5° C. protected from light until required. When required, the proteins were transferred to acrylic slides (VACR) using a Genetix Q-Array pin arrayer (Genetix Ltd.).

Transfer of un-derivatized proteins and/or derivatized proteins to acrylic slides: The Genetix Q-Array pin arrayer was used to transfer un-derivatized and/or derivatized protein/polymeric matrix solution to an acrylic slide via 300 μm pins. After deposition was complete, the slides were transferred to an isolation chamber for re-equilibration under saturated polymeric matrix atmosphere for 30 min, after which the isolation chamber was placed in an ultraviolet (UV) cabinet for 30 min under nitrogen gas to remove existing oxygen in the chamber in order to ensure complete polymerization. Slides were then removed and stored sealed and protected from light and dust.

### Example 2

This example describes the preparation of another GDPB.

Culture of Y. pestis KIM D27: An attenuated strain of Y. pestis designated KIM D27 was obtained from the laboratory of Dr. Olaf Schneewind at the University of Chicago, Chicago, Ill. Approximately 2 ml of Heart Infusion Broth (HIB) were inoculated by loop from a 15% glycerol stock (−80° C.) of Y. pestis KIM D27 and incubated overnight at 27° C. on a roller drum. Cell density was monitored with a Biocrom WPA CO8000 Cell Density Meter to ensure cells were in log phase before induction. Induction of surface virulence proteins (type III secretion apparatus) was accomplished by inoculating 4 ml of Ca²⁺-deficient medium (1:20 in TMH) from the HIB log-phase culture. The culture was allowed to incubate for 2 hr at 27° C., followed by a 4 hr incubation at 37° C., at which time the culture was harvested. The combination of low Ca²⁺ (<2 mM) and 37° C. growth conditions induces Y. pestis to express virulence proteins. Cells were washed three times with cold PBS prior to lysis.

Bacterial lysis: Two ml of lysis buffer (6 M urea, 2 M thiourea, 10% glycerol, 50 mM Tris-HCl, 2% n-octylglucoside, 5 mM TCEP, 1 mM protease inhibitor) were added to 0.5 ml of cell pellet, and the mixture was vortexed aggressively and allowed to incubate 30 min at RT. Six freeze-thaw cycles consisting of 3 min in dry ice/acetone followed by thawing at 42° C. and vortexing was performed. The lysate was centrifuged at 8,000 g for 60 min, and the supernatant was decanted and stored at −80° C. until fractionation.

PF2D: Y. pestis KIM D27 lysate was fractionated. A PD-10 column (Amersham Biosciences, Piscataway, N.J.) was equilibrated with approximately 25 ml of chromatofocusing (CF) start buffer (6 M urea, 0.1% n-octyl glucoside, 25 mM triethanolamine). The supernatant was loaded onto the PD-10 column. The proteins were eluted from the PD-10 column using CF start buffer, collecting the first 3.5 ml fraction of the eluent. Three ml of the fraction were injected onto the HPCF column and analyzed according to the ProteoSep protocol (Eprogen, Inc., Darien, Ill.). After isoelectric focusing, the resultant samples were applied to a NPS-RP-HPLC for fractionation based on hydrophobicity. An automated fractionater deposited 500 μl per well in nine 96-deep well plates.

Transfer of un-derivatized proteins and/or derivatized proteins to acrylic slides: The Genetix Q-Array pin arrayer was used to transfer un-derivatized and/or derivatized protein/polymeric matrix solution to an acrylic slide as described in Example 1.

### Example 3

This example describes an immunoassay for Streptococcus using a biochip fabricated with derivatized proteins.

Slides were blocked overnight at 5° C. with PBS containing 1% bovine serum albumin (BSA). Slides were washed in PBST for 5 min, rinsed with de-ionized water, and dried with filtered air. The slides were then incubated with approximately 35 μl of Streptococcus (1×10⁴) in PBST with 1% BSA for 7 min at RT, and then rinsed with PBST for 1 min. Afterwards, the slides were incubated with approximately 35 μl of 1:100 Alexa 568-labeled detection antibody in PBST with 1% BSA for 6 min at RT, while protected from light. After rinsing for 1 min in PBST, the slides were rinsed with de-ionized water and dried with filtered air, while protected from light. Afterwards, the slides were imaged on the Aurora Photonic Imager (Aurora Photonics, Inc., Barrington, Ill.). An exposure time of 5 sec was used. The presence of Streptococcus at a concentration of 1×10⁴ microbes/ml was easily detected.

### Example 4

This example describes an immunoassay for Y. pestis proteins using a biochip fabricated with un-derivatized proteins.

Slides were blocked overnight at 5° C. with PBS containing 1% BSA. Slides were washed in PBST for 5 min, rinsed with de-ionized water, and dried with filtered air. The slides were then incubated with approximately 200 μl of a 1:100 dilution of anti-Caf1 (Caf1 is a virulence protein found on the surface of Y. pestis; antibody provided by Olaf Schneewind, University of Chicago) in PBST with 1% BSA for 90 min at RT, and then rinsed with de-ionized water and placed in 0.05% PBST for 30 min at RT. Afterwards, the slides were incubated with Alexa 594-labeled protein A for 90 min at RT. After rinsing with deionized water, the slides were placed in 0.05% PBST for 30 min at RT. The slides were then rinsed with de-ionized water and dried with filtered air, while protected from light. Afterwards, the slides were imaged on the Aurora Photonic Imager (Aurora Photonics, Inc., Barrington, Ill.). An exposure time of 5 sec was used. The Y. pestis fractions reacted with anti-Caf1 antibody. Control proteins Caf1 and LcrV also reacted.

All references, including publications, patent applications, and patents, cited herein are hereby incorporated by reference to the extent that they relate to materials and methods described herein.

The use of the terms “a,”“an,” “the,” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illuminate better the invention and does not pose a limitation on the scope of the invention unless otherwise claimed. No language in the specification should be construed as indicating any non-claimed element as essential to the practice of the invention.

What is claimed is:

1. A method of making a gel drop protein chip, which method comprises transferring proteins, which were obtained from a cellular lysate, partitioned using protein fractionation (PF2D), and mixed with a polymeric matrix solution comprising acrylamide/bis and glycerol, to an array, whereupon a gel drop protein chip is made.

2. The method of claim 1, wherein at least some of the proteins are derivatized with N-hydroxysuccinimide ester of N-methacryloyl-6-aminocaproic acid (NHS monomer) prior to being mixed with the polymeric matrix solution comprising acrylamide/bis and glycerol.

3. The method of claim 1, wherein the PF2D comprises partitioning on the basis of isoelectric focusing, and fractionating on the basis of hydrophobicity using reverse phase-separation in liquid phase.

4. The method of claim 2, wherein the PF2D comprises partitioning on the basis of isoelectric focusing, and fractionating on the basis of hydrophobicity using reverse phase-separation in liquid phase.

5. The method of claim 1, wherein the polymeric matrix solution comprises 5% acrylamide/bis and 65% glycerol (w/v) in 35 mM sodium phosphate, pH 7.25.

6. The method of claim 2, wherein the polymeric matrix solution comprises 5% acrylamide/bis and 65% glycerol (w/v) in 35 mM sodium phosphate, pH 7.25.

7. The method of claim 2, wherein the proteins are derivatized with about a 10-fold excess of a 0.5% solution of the NHS monomer in N,N-dimethylformamide (DMF).

8. A method of making a gel drop protein chip, which method comprises transferring proteins, which were derivatized with NHS monomer, and mixed with a polymeric matrix solution comprising acrylamide/bis and glycerol, to an array, whereupon a gel drop protein chip is made.

9. The method of claim 8, wherein the polymeric matrix solution comprises 5% acrylamide/bis and 65% glycerol (w/v) in 35 mM sodium phosphate, pH 7.25.

10. The method of claim 8, wherein the proteins are derivatized with about a 10-fold excess of a 0.5% solution of the NHS monomer in DMF.

11. A gel drop protein chip comprising proteins in a polymeric matrix solution comprising acrylamide/bis and glycerol.

12. The gel drop protein chip of claim 11, wherein the polymeric matrix solution comprises 5% acrylamide/bis and 65% glycerol (w/v) in 35 mM sodium phosphate, pH 7.25.

13. The gel drop protein chip of claim 11, wherein the proteins are derivatized with NHS monomer.

14. The gel drop protein chip of claim 13, wherein the proteins were derivatized with about a 10-fold excess of a 0.5% solution of the NHS monomer in DMF.

15. The gel drop protein chip of claim 13, wherein the polymeric matrix solution comprises 5% acrylamide/bis and 65% glycerol (w/v) in 35 mM sodium phosphate, pH 7.25.

16. A method of using the gel drop protein chip of claim 11, which method comprises using the gel drop protein chip to interrogate a sample.

17. A method of using the gel drop protein chip of claim 13, which method comprises using the gel drop protein chip to interrogate a sample.

18. A protein derivatized with NHS monomer.
